(19) United States
(12) Patent Application Publication
(10) Pub. No.: US 2008/0281285 A1
(43) Pub. Date: Nov. 13, 2008

(54) Process for Bonding a Material to a Substrate with an Adhesive Precursor Forming an Adhesive as a Product of Ester Condensation and Products Bonded with Such Adhesive Precursor

(76) Inventors: Isao Noda, Fairfield, OH (US); William Maxwell Allen, JR., Liberty Twp., OH (US); James Terry Knapmeyer, Rossmoyne, OH (US); Michael Matthew Satkowski, Oxford, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/961,404

(22) Filed: Dec. 20, 2007

Related U.S. Application Data
(60) Provisional application No. 60/928,740, filed on May 11, 2007.

(51) Int. Cl. A61F 13/15; B32B 27/00

(52) U.S. Cl. 604/365; 156/332; 428/411.1; 604/367; 604/385.29

(57) ABSTRACT

A process is provided for bonding a substrate to a material using an adhesive precursor comprising a reactive mixture which is reacted to form an adhesive as a product of ester condensation. The reactive mixture includes a monomer mixture comprising at least one polyhydric alcohol and a reactant selected from the group consisting of at least one organic polyacid; at least one organic anhydride; and combinations thereof. Alternatively, the reactive mixture comprises a prepolymer formed from the monomer mixture; a combination of the prepolymer and the monomer mixture; or a combination of the prepolymer and reactants such as polyhydric alcohol, organic polyacid, organic anhydride, and combinations thereof.

### CROSS REFERENCE TO RELATED APPLICATION

This application claims the benefit of provisional Application No. 60/928,740, filed May 11, 2007.

### FIELD OF THE INVENTION

The present invention is directed to a reactive mixture comprising either a monomer mixture or reactive prepolymer capable of making crosslinked thermoset resins, particularly alkyd resins, formulated to adhesively bond a substrate. In a specific embodiment, the reactive mixture provides an adhesive precursor which is applied to a substrate and reacted to form an adhesive as a product of ester condensation to bond the substrate to a material.

### BACKGROUND OF THE INVENTION

Adhesives are used in many applications for bonding articles and materials. In particular, disposable articles such as sanitary napkins, catamenials and diapers require the use of adhesives to join individual components making up the disposable article or to form such components by bonding substrates, such as nonwovens, to other substrates or materials.

Adhesive is typically applied as a liquid. In the liquid form, the adhesive wets and flows into the crevices of the adherend. The liquid form of the adhesive is obtained by heating to the point that flow occurs or dissolving or dispersing the material in a solvent. The adhesive then undergoes a phase change to a solid either by cooling, solvent evaporation, or reaction, in order for the joint to acquire the necessary strength to resist shearing forces.

Alkyd is a term applied to a group of synthetic thermoset resins best described as polyester condensate resins. This group of material comprises ester condensates of polyhydric alcohols and organic polyacids. Glycerin is the predominant polyhydric alcohol component used in ester condensates. An increasing supply of glycerin has prompted the opportunity for developing applications utilizing alkyd resins.

It is known to use alkyd resins or polymers in combination with solvents, plasticizers and other ingredients to form adhesives. Such adhesives are typically viscous and tacky making them difficult to handle during application. However, alkyd resins start as a low viscosity liquid reactive mixture comprising either a monomer mixture or reactive prepolymer mixture which can be formulated in a free flowing liquid state which is easy to apply. Such monomer or prepolymer reactive mixture can be cured by a crosslinking chemical reaction typically induced by the application of heat to form a viscous or hard bonding material.

The need exist for a process for bonding substrates or materials starting with liquid monomer or prepolymer reactive mixtures that polymerize or react forming an adhesive as a product of ester condensation subsequent to application.

### SUMMARY OF THE INVENTION

The present invention provides a process for bonding a substrate to a material with an adhesive precursor comprising a reactive mixture. The reactive mixture includes a monomer mixture comprising at least one polyhydric alcohol and a reactant selected from the group consisting of at least one organic polyacid; at least one organic anhydride; and combinations thereof. Alternatively, the reactive mixture comprises a prepolymer formed from the monomer mixture; a combination of the prepolymer and the monomer mixture; or a combination of the prepolymer and reactants such as polyhydric alcohol, organic polyacid, organic anhydride, and combinations thereof. The adhesive precursor is reacted to form an adhesive as a product of ester condensation.

The invention is also directed to a composite comprising a substrate, a material and an adhesive precursor which is reacted to form an adhesive as a product of ester condensation bonding the substrate and the material. The adhesive precursor comprises a reactive mixture including a monomer mixture comprising at least one polyhydric alcohol and a reactant selected from the group consisting of at least one organic polyacid; at least one organic anhydride; and combinations thereof. Alternatively, the reactive mixture comprises a prepolymer formed from the monomer mixture; a combination of the prepolymer and the monomer mixture; or a combination of the prepolymer and reactants such as polyhydric alcohol, organic polyacid, organic anhydride, and combinations thereof.

The invention is further directed to articles and packaging comprising at least two components and the aforementioned adhesive precursor disposed therebetween which is reacted to form an adhesive as a product of ester condensation to join the at least two components.

### BRIEF DESCRIPTION OF THE DRAWINGS

FIG. 1 is a plan view of a body-facing surface of a disposable diaper showing various components that may be joined using the adhesive precursor of the present invention.

### DETAILED DESCRIPTION OF THE INVENTION

All percentages, ratios and proportions used herein are by weight percent of the reactive mixture, unless otherwise specified. All average values are calculated “by weight” of the reactive mixture or components thereof, unless otherwise expressly indicated. “Average molecular weight,” or “molecular weight” for polymers, unless otherwise indicated, refers to weight average molecular weight. Weight average molecular weight, unless otherwise specified, is determined by gel permeation chromatography.

“Copolymer” as used herein is meant to encompass copolymers, terpolymers, and other multiple-monomer polymers.

“Reactant” as used herein refers to a chemical substance that is present at the start of a chemical reaction and reacts with one or more other substances or catalysts in or exposed as part of a chemical reaction.

“Mixture” as used herein refers to a mixture of two or more of any of a defined group of components, unless otherwise specified. Lists of alternative ingredients include mixtures of such ingredients unless otherwise specified.

“Biodegradable” as used herein refers to the ability of a compound to ultimately be degraded completely into CH₄, CO₂ and water or biomass by microorganisms and/or natural environmental factors.

“Compostable” as used herein refers to a material that meets the following three requirements: (1) the material is capable of being processed in a composting facility for solid waste; (2) if so processed, the material will end up in the final compost; and (3) if the compost is used in the soil, the material will ultimately biodegrade in the soil.

“Comprising” as used herein means that various components, ingredients or steps can be conjointly employed in practicing the present invention. Accordingly, the term “comprising” encompasses the more restrictive terms “consisting essentially of” and “consisting of”. The present reactive compositions can comprise, consist essentially of, or consist of any of the required and optional elements disclosed herein.

“Adhesive” as used herein means a material that joins two other materials, called adherends, together.

Markush language as used herein encompasses combinations of the individual Markush group members, unless otherwise indicated.

Regarding all numerical ranges disclosed herein, it should be understood that every maximum numerical limitation given throughout this specification includes every lower numerical limitation, as if such lower numerical limitations were expressly written herein. In addition, every minimum numerical limitation given throughout this specification will include every higher numerical limitation, as if such higher numerical limitations were expressly written herein. Further, every numerical range given throughout this specification will include every narrower numerical range that falls within such broader numerical range and will also encompass each individual number within the numerical range, as if such narrower numerical ranges and individual numbers were all expressly written herein.

The present reactive mixture, processes and articles employ adhesives comprising a reactive mixture capable of making crosslinked thermoset resins, particularly alkyd resins, from an ester condensation reaction. The reactive mixture comprises a monomer mixture including polyhydric alcohol and a polyfunctional organic polyacid or anhydride. The reactive mixture can also include a prepolymer made by reacting the monomer mixture to a precrosslinking stage, or a combination of the prepolymer and the monomer. The reactive mixture is formulated to be easily applied to a substrate surface as a free flowing liquid adhesive precursor which can be reacted to form an adhesive. During the reaction, the adhesive precursor can be heated to an elevated temperature sufficient to induce an ester condensation reaction of the reactive mixture which polymerizes and crosslinks the mixture by liberating water as a reaction byproduct to open atmosphere resulting in a hard bonding material.

The materials used in forming the aforementioned adhesive precursor, methods of making the same and articles formed utilizing the adhesive precursor are further discussed below.

### Polyhydric Alcohol

The reactive mixture used in forming the adhesive precursor includes polyhydric alcohol. “Polyhydric alcohol” as used herein refers to an alcohol having two or more alcohol (i.e., hydroxyl) functional groups. Any suitable polyhydric alcohol or combination of polyhydric alcohols is of use; however, monomers, oligomers, or short chain polymer polyhydric alcohols having a molecular weight of less than 2000 g/mol are preferred. Non-limiting examples of suitable polyhydric alcohols include: glycerol (also known in the art as glycerin), glycol, sugar, sugar alcohol, and combinations thereof. Non-limiting examples of glycols of use include: ethylene glycol, propylene glycol, dipropylene glycol, butylene glycol, hexane triol, and the like, oligomers thereof, and combinations thereof. Non-limiting examples of sugars of use include: glucose, sucrose, fructose, raffinose, maltodextrose, galactose, xylose, maltose, lactose, mannose, erythrose, pentaerythritol, and mixtures thereof. Non-limiting examples of sugar alcohols of use include: erythritol, xylitol, malitol, mannitol, sorbitol, and mixtures thereof. In specific embodiments of the present invention, the polyhydric alcohol comprises glycerol, mannitol, sorbitol, and combinations thereof.

Another form of polyhydric alcohol suitable in forming the reactive mixture includes crude glycerin. Crude glycerin is derived from various reactions of a triglyceride which is basically glycerin and three fatty acids linked together by ester bonds. Reactions which generate crude glycerin include esterification, hydrolysis, and saponification. Crude glycerin is typically 80-95% glycerin and contains some level of water (moisture), typically 3-15%, based on the chemistry and recovery process. Crude glycerin will also contain some level of non-glycerin organics, quantified as total fatty acid. These are typically unreacted triglycerides (or diglycerides/monoglycerides), fatty acids, and methyl esters.

Typically, the polyhydric alcohol can be present in reactive mixtures of the present invention in an amount of from about 5% to about 80%, from about 10% to about 75%, from about 25% to about 70%, or from about 35% to about 65%.

### Organic Polyacid and Anhydrides

The reactive mixture used in forming the adhesive precursor also includes organic polyacids and anhydrides. The organic polyacid means an organic acid having two or more acid functionalities and can include, but is not limited to, diacids, triacids (having at least three acid groups), other acids with four or more acid functionalities, acid polymers or copolymers, or mixtures thereof. Such acids include, but are not limited to adipic acid, sebatic acid, citric acid, oxalic acid, malonic acid, succinic acid, glutaric acid, maleic acid, fumaric acid, phthalic acid, isophthalic acid, terphthalic acid, and mixtures of two or more thereof. Anhydrides of such acids may also be employed and within the context of the present specification, reference to organic polyacid includes such anhydrides. Monoacids such as lauric acid, stearic acid, myristic acid, palmitic acid, oleic acid, linoleic acid, sebacic acid, acrylic acid, methacrylic acid, itaconic acid, and glycidyl methacrylate may optionally be included in addition to polyacids at any stage. For example, monoacids may be added as processing aids or to modify properties of the final product, e.g. flexibility, strength, etc.

For the present invention many different types of organic polyacids and anhydrides can be used including adipic acid, citric acid, maleic acid, maleic anhydride, polyacrylic acid, phthalic anhydride, and the like, as well as their mixtures. Monobasic acids, especially fatty acids like stearic acid, lauric acid, oleic acid, and linoleic acid, can also be incorporated into the reactive mixture. Other functional compounds with reactive acid or alcohol functionality, such as oligomeric silicone or polyethylene glycol, may also be incorporated.

Typically, the organic polyacid or anhydride is employed in the reactive mixtures of the present invention in an amount of from about 5% to about 80%, from about 10% to about 75%, from about 25% to about 70%, or from about 35% to about 65%.

### Triglyceride

In addition to triglycerides associated with crude glycerin described above, suitable triglycerides, which are also known in the art as triglycerols, may be included in the reactive mixture. Non-limiting examples of triglycerides of use include: tristearin, triolein, tripalmitin, 1,2-dipalmitoolein, 1,3-dipalmitoolein, 1-palmito-3-stearo-2-olein, 1-palmito-2-stearo-3-olein, 2-palmito-1-stearo-3-olein, trilinolein, 1,2-dipalmitolinolein, 1-palmito-dilinolein, 1-stearo-dilinolein, 1,2-diacetopalmitin, 1,2-distearo-olein, 1,3-distearo-olein, trimyristin, trilaurin and combinations thereof.

Suitable triglycerides may be added to the present reactive compositions in neat form. Additionally, or alternatively, oils and/or processed oils containing suitable triglycerides may be added to the reactive compositions. Non-limiting examples of oils include coconut oil, corn germ oil, olive oil, palm seed oil, cottonseed oil, palm oil, rapeseed oil, sunflower oil, whale oil, soybean oil, peanut oil, linseed oil, tall oil, and combinations thereof.

Typically, triglycerides are employed in the reactive mixture in an amount up to about 75%, or from about 2% to about 50%, or from about 5% to about 25%.

In some embodiments, combinations of acid and triglyceride are employed in the reactive mixture. In such embodiments, the total amounts of acid and triglyceride is from about 20% to about 80%, from about 30% to about 70%, or from about 40% to about 60%. Additionally, or alternatively, the molar ratio of the alcohol functional groups to the total of ester and acid functional groups is at least about 1:1, or at least about 4:1. In some embodiments, the molar ratio is from about 1:1 to about 200:1, or from about 1:1 to about 50:1.

The reactive mixture of the present invention may also include monobasic acid, and appropriate amounts of monoglyceride, or diglyceride as alternatives to triglyceride.

### Additional Components

The reactive mixtures used in forming the adhesive precursor may further include one or more additional components as desired for processing and/or end use of the composition. Additional components may be present in any suitable amount. In some embodiments, additional components may be present in an amount of from about 0.01% to about 35% or from about 2% to about 20% by weight of the reactive mixture. Non-limiting examples of additional components include, but are not limited to, additional polymers, processing aids and the like.

Non-limiting examples of additional polymers of use include: polyhydroxyalkanoates, polyethylene, polypropylene, polyethylene terephthalate, maleated polyethylene, maleated polypropylene, polylactic acid, modified polypropylene, nylon, caprolactone, and combinations thereof. Additional polymers also include polyvinyl alcohol and polyhydric alcohols having molecular weights of greater than 2000 g/mol.

In embodiments in which properties including, but not limited to, biodegradability and/or flushability are desired, additional suitable biodegradable polymers and combinations thereof are of use. In some embodiments, polyesters containing aliphatic components are suitable biodegradable thermoplastic polymers. In some embodiments, among the polyesters, ester polycondensates containing aliphatic constituents and poly(hydroxycarboxylic acid) are preferred. The ester polycondensates include, but are not limited to: diacids/diol aliphatic polyesters such as polybutylene succinate, and polybutylene succinate co-adipate; aliphatic/aromatic polyesters such as terpolymers made of butylenes diol, adipic acid, and terephthalic acid. The poly(hydroxycarboxylic acids) include, but are not limited to: lactic acid based homopolymers and copolymers; polyhydroxybutyrate; and other polyhydroxyalkanoate homopolymers and copolymers. In some embodiments, a homopolymer or copolymer of poly lactic acid is preferred. Modified polylactic acid and different stereo configurations thereof may also be used. Suitable polylactic acids typically have a molecular weight range of from about 4,000 g/mol to about 400,000 g/mol. Examples of suitable commercially available poly lactic acids include NATUREWORKS™ from Cargill Dow and LACEA™ from Mitsui Chemical. An example of a suitable commercially available diacid/diol aliphatic polyester is the polybutylene succinate/adipate copolymers sold as BIONOLLE™ 1000 and BIONOLLE™ 3000 from the Showa Highpolmer Company, Ltd. Located in Tokyo, Japan. An example of a suitable commercially available aliphatic/aromatic copolyester is the poly(tetramethylene adipate-co-terephthalate) sold as EASTAR BIO™ Copolyester from Eastman Chemical or ECOFLEX™ from BASF. In some embodiments, the biodegradable polymer or combination of polymers may comprise polyvinyl alcohol.

The aforementioned biodegradable polymers and combinations thereof may be present in an amount of from about 0.1% to about 70%, from about 1% to about 50%, or from about 2% to about 25%, by weight of the reactive mixture.

Processing aids are generally present in the reactive mixture in amounts of from about 0.1% to about 3% or from about 0.2% to about 2% by weight of the reactive mixture. Non-limiting examples of processing aids include: lubricants, anti-tack, polymers, surfactants, oils, slip agents, and combinations thereof. Non-limiting examples of specific processing aids include: Magnesium stearate; fatty acid amides; metal salts of fatty acids; wax acid esters and their soaps; montan wax acids, esters and their soaps; polyolefin waxes; non polar polyolefin waxes; natural and synthetic paraffin waxes; fluoro polymers; and silicon. Commercial examples of such compounds include, but are not limited to: Crodamide™ (Croda, North Humberside, UK), Atmer™ (Uniqema, Everberg, Belgium,) and Epostan™ (Nippon Shokobai, Tokyo, JP).

Other additives can be present in the reactive mixture to impart additional physical properties to the final product or material formed therefrom. Such additives include compounds having functional groups such as acid groups, alcohol groups and combinations thereof. Such compounds include oligomeric silicone, polyethylene glycol and combinations thereof.

### Fillers

The fillers can be mixed with the reactive mixture providing the adhesive precursor. Fillers comprise solid particulates having an equivalent diameter of less than 300 microns, less than 100 microns or less than 50 microns. Non-limiting examples of fillers present in the reactive mixture of the present invention include: talc, clay, pulp, wood, flour, walnut shells, cellulose, cotton, jute, raffia, rice chaff, animal bristles, chitin, TiO₂, thermoplastic starch, raw starch, granular starch, diatomaceous earth, nanoparticles, carbon fibers, kenaf, silica, inorganic glass, inorganic salts, pulverized plasticizer, pulverized rubber, polymeric resins and combinations thereof. Further additives including inorganic fillers such as the oxides of magnesium, aluminum, silicon, and titanium may also be added as inexpensive fillers or processing aides. Other inorganic materials include hydrous magnesium silicate, titanium dioxide, calcium carbonate, boron nitride, limestone, mica glass quartz, and ceramics. Additionally, inorganic salts, including alkali metal salts, alkaline earth metal salts, phosphate salts, may be used as processing aides. Another material that can be added is a chemical composition formulated to further accelerate the environmental degradation process such as cobalt stearate, citric acid, calcium oxide, and other chemical compositions found in U.S. Pat. No. 5,854,304 to Garcia et al.

The aforementioned fillers and combinations thereof may be present in the reactive mixture forming the adhesive precursor in an amount up to about 40% by weight of the reactive mixture; from about 1% to about 30%, 2% to about 20%, and 5% to about 10%, by weight of the reactive mixture.

### Ester Condensation Reaction

As previously described herein, an adhesives comprising an alkyd resin are made from the condensation reaction of a reactive mixture comprising monomers, such as polyhydric alcohol and a polyfunctional organic polyacid, or from an oligomer which is a prepolymer made by reacting the monomer mixture to a precrosslinking stage where condensation reaction has already at least partially, but not completely taken place between the polyhydric alcohol and the acid. During the condensation reaction, if the temperature of the reactive mixture is sufficiently high and for a sufficient time to drive a reaction between the polyhydric alcohol and the acid, the composition which is formed will convert to a water stable alkyd resin composition. For example, the reactive mixture can be processed providing sufficient removal of water for conversion to a water stable composition. In such an embodiment, the composition can be processed to a form which is suitable for end use such as cured adhesives bonding substrates, materials, disposable article components and combinations thereof.

On the other hand, if the temperature or conditions at which the melt processing of the reactive mixture is conducted is sufficiently low and/or for an insufficient time to drive reaction between the polyhydric alcohol and the acid, the resulting extrudate comprises a reactive mixture, which may be further processed, if desired, and which is convertible to water stable compositions by further heating. The reactive mixture can therefore be provided in this embodiment in a liquid form which can be applied to a surface of a substrate in the form of an adhesive precursor which can be subjected to sufficient conditions of temperature and time to effect the conversion of the reactive composition to a water stable adhesive composition. Alternatively, if the adhesive precursor is not subjected to sufficient conditions of temperature and time to effect the conversion of the reactive composition to a water stable adhesive composition, the resulting reactive composition can be subsequently heated and converted to a water stable adhesive.

### Adhesive Precursor Application

Any suitable applicator may be used to apply the adhesive precursor to a material or substrate such as: a printing station (such as rotogravure or flexographic for example), a spraying station,), a coater station (such as slot, roll, or air knife for example), a size press station, or a foam applicator station. A suitable apparatus for applying the adhesive precursor is disclosed in U.S. Pat. No. 5,840,403 issued to Trokhan et al. on Nov. 24, 1998, and herein incorporated by reference.

Any known printing technique can be used to apply the adhesive precursor including gravure printing, offset printing, flexographic printing, slot coating, ink jet printing (e.g., thermal drop on demand ink jets, piezoelectric drop on demand ink jets, continuous ink jets, etc.), and other forms of digital printing including electrostatic printing and electrophotography, such as the CreoScitex SP system of CreoScitex (Tel Aviv, Israel). Other exemplary printer systems include the Vutek UltraVu printers (Vutek, Meredith, N.H.) as examples of high resolution, wide ink jet printers (2 me-ters, for example); the DisplayMaker FabriJet XII 12-cartridge printer of Color Span Corp. (Eden Prairie, Minn.), and the wide ink-jet printing Artistri system of DuPont (Wilmington, Del.). Printing techniques conventionally used for applying inks can generally be adapted to apply adhesive precursors with or without added color. For example, principles of adapting flexographic printing for the application of adhesive precursors to tissue and other fibrous webs has been disclosed in U.S. application Ser. No. 10/329,991, “Flexographic Printing to Deliver Highly Viscous Agents in a Pattern to the Skin-Contacting Surface of an Absorbent Article,” filed Dec. 26, 2002, by Chen and Lindsay, and in U.S. application Ser. No. 10/305,791, “Structural Printing of Absorbent Webs,” filed Nov. 27, 2002, by Chen et al., both of which are herein incorporated by reference. Anilox rolls for application of printed adhesive to one or both sides of a tissue web are disclosed in U.S. Pat. No. 6,607,630, “Print Bonded Multi-Ply Tissue,” issued Aug. 19, 2003, to Bartman et al., which can be adapted for printing single or multi-ply webs.

Any known spray technology can be used to apply the adhesive precursor, including DRYAD spray technology by Dryad Technology, Delaware, as described by R. H. Donnelly and M. Kangas, Paperi ja Puu, Vol. 83, No. 7, pp. 530-531. Another embodiment is disclosed in U.S. Pat. No. 4,944,960, “Method and Apparatus for Coating Paper and the Like,” issued Jul. 31, 1990 to Sundholm et al. In this technology, the adhesive precursor passes into a nozzle that ejects the material to a region with an annular high-velocity gas flow around it that carries the precursor material to the surface of substrate. Electrostatic charge can be used to improve delivery of the adhesive precursor to the substrate. Printing of the adhesive precursor can be done selectively or uniformly to a surface of a substrate.

The adhesive precursor can be applied in any desirable pattern such as fine lines, dots, crossing lines, sinuous lines, patterns that form recognizable images such as those of flowers or other patterns. In various embodiments of the invention, the adhesive precursor occupies from about 15% to about 60% of the surface area of one side of a substrate. Alternatively, the adhesive precursor may occupy any of the following percentage ranges of one side of a substrate: about 5% or more, about 30% or more, over 50%, from about 10% to about 90%, from about 20% to about 80%, from about 20% to about 70%, less than about 60%, and less than 50%.

### Adhesive Precursor Post Curing

When the adhesive precursor formed from the reactive mixtures are applied to a surface of a substrate to be adhesively bonded, the crosslinking reaction can be completed either during the application of the adhesive precursor or by an additional post curing step. In order to produce fully crosslinked adhesive from the adhesive precursor, the ester condensation reaction of the reactive mixture is induced, and/or driven towards completion through the application of heat. Water produced as a reaction byproduct is effectively removed to promote the reaction. The reaction mixture temperature may be between about 100° C. and about 300° C., between about 120° C. and about 280° C., or between about 150° C. and about 260° C. to drive the crosslinking reaction to completion. In some embodiments of the present invention, a catalyst may be used to initiate and/or accelerate the ester condensation and/or transesterification reactions. Any suitable catalyst is of use. Non-limiting examples of useful catalysts include Lewis acids. A non-limiting example of a Lewis acid is para-toluene sulfonic acid.

Completing the crosslinking reaction via post curing can be accomplished in a conventional convective or radiant oven or microwave oven, as well as other means to heat the adhesive precursor during the post curing step to complete the ester condensation reaction and corresponding final removal of water from the article.

### Articles

As used herein, “article” is meant to encompass articles having at least one portion joined with an adhesive precursor according to the present invention. Articles include, but are not limited to disposable articles and packaging. Disposable articles include adult incontinence products, feminine hygiene pads and sanitary napkins, disposable diapers and training pants. Packaging formats include flexible bags, semi-flexible bags, rigid bags, pouches, carton or plastic boxes, canisters, bottles, tubes, and combinations thereof. For particular applications, the packaging is selected in accordance with the product being contained and/or consumer preferences.

### Disposable Personal Care Products

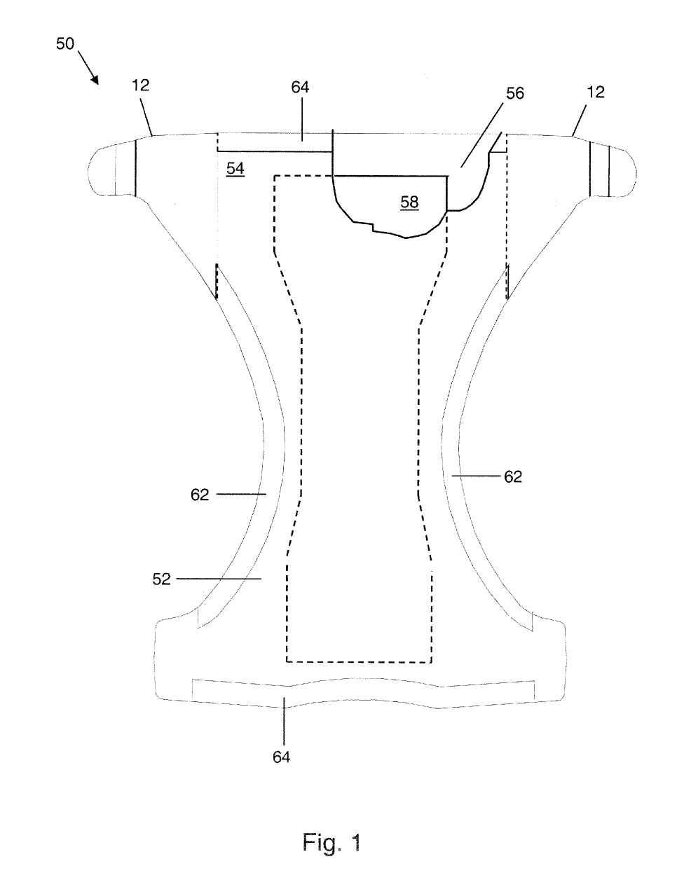

The present invention can be used for disposable personal care products comprising components joined via adhesive precursors comprising reactive mixtures of the present invention. In some embodiments, disposable personal care absorbent articles comprise a liquid pervious topsheet, a liquid impervious backsheet, an absorbent core positioned between the topsheet and backsheet, as well as other components which can be joined using the adhesive precursor of the present invention. An example of such disposable personal care product is a disposable diaper 50 shown in FIG. 1 in a flat-out state with the portion of the diaper 50 which faces the wearer oriented towards the viewer. As shown in FIG. 1, portions of the structure are cut-away to more clearly show the construction of the diaper 50. The diaper 50 comprises a liquid pervious topsheet 54; a liquid impervious backsheet 56; an absorbent core 58 which is preferably positioned between at least a portion of the topsheet 54 and the backsheet 56; extensible leg cuffs 62, and elastic waist features 64. The chassis 52 of the diaper 50 comprises the main body of the diaper 50 and includes the topsheet 54 and/or the backsheet 56 and at least a portion of the absorbent core 58. While the topsheet 54, the backsheet 56, the absorbent core 58, fastening members 12 and other aforementioned constituents may be assembled in a variety of well known configurations and bonded using the adhesive precursor of the present invention, preferred diaper configurations are described generally in U.S. Pat. No. 3,860,003 entitled “Contractible Side Portions for Disposable Diaper” issued to Kenneth B. Buell on Jan. 14, 1975; U.S. Pat. No. 5,151,092 issued to Buell on Sep. 9, 1992; and U.S. Pat. No. 5,221,274 issued to Buell on Jun. 22, 1993; and U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; U.S. Pat. No. 5,580,411 entitled “Zero Scrap Method for Manufacturing Side Panels for Absorbent Articles” issued to Nease et al. on Dec. 3, 1996; and U.S. Pat. No. 6,004,306 entitled “Absorbent Article with Multi-Directional Extensible Side Panels” issued to Robles et al. on Dec. 21, 1999.

### Packaging

The present invention can also be used for packaging comprising components joined via adhesive precursors comprising reactive mixtures of the present invention. For instance, fully enclosed cartons that provide protection for the containers housed therein are formed from a blank into a carton having top, side and bottom panel components. The carton is folded and joined along the bottom panels, using the adhesive precursor of the present invention which is reacted to form an adhesive as a product of ester condensation. The carton pack is filled with product containers and then turned 90° where the side doors of the carton end are closed. The top panel is then forced downward and is joined to the side doors via the adhesive precursor and aforementioned ester condensation reaction. The adhesive precursor is also applicable to flexible packaging such as bags or pouches having open end components where adhesive precursor is applied to the open ends and reacted to form an adhesive as a product of ester condensation thereby bonding the open ends together.

### EXAMPLES

### Example 1

### Preparation of Glycerol-Maleate Oligomer Adhesive Precursor

To a beaker, 92.09 g glycerol (P&G Chemicals, Cincinnati, Ohio), one mole, and 0.48 g p-toluenesulfonic acid (Aldrich, Milwaukee, Wis.) is added. The beaker is placed on a plate situated under an overhead stirrer fitted with a 4-blade paddle mixing implement and a Brookfield viscometer (Middleboro, Mass.). The glycerol p-toluenesulfonic acid mixture is stirred and heated to 60° C. One mole of maleic anhydride (Aldrich, Milwaukee, Wis.), 98.06 g, is slowly added to the glycerol p-toluenesulfonic acid mixture while stirring. The mixture temperature is slowly raised 80° C. until a clear slightly straw-colored solution is formed. The temperature is raised to 140° C. Some bubbling is noticeable at this time. The solution is stirred at 140° C. until a viscosity of 2 poise is indicated by the viscometer. The material is clear and straw-colored and easily poured.

### Example 2

### Use of Glycerol Maleate Oligomer as an Adhesive for Paper

To test adhesive properties a simple adhesive test is completed. The samples for the test are prepared in accordance with ASTM standard D1876-01, “Peel Resistance of Adhesives.” Samples consist of two sheets of paper stock of weight 180 g/m² cut into strips 305 mm long and 25 mm wide. A thin layer of the oligomer adhesive precursor of example 1 is spread on one of the strips of paper. The adhesive precursor is uniformly spread on 241 mm of the paper measured from one end of the strip. The other paper strip is pressed onto the coated strip, spanning the length of the coating. The amount of the adhesive spread on the bonded area is 0.3 g. Ten samples are made. The samples are then cured in a convection oven at 130° C. for four hours. After curing the samples are allowed to cool to room temperature and conditioned at room temperature for 12 hours. The bonded strips of paper are pulled apart by an Instron tester (Norwood, Mass.) set at a constant head speed of 254 mm/min. In all ten cases the paper is torn before the adhesive fails, indicating cohesive failure and showing that the strength of the adhesive is greater than the substrate. This makes for a sufficient adhesive material for package construction.

### Example 3

### Use of Glycerol Maleate Oligomer as an Adhesive for Polymers

To test adhesive properties a simple adhesive test is completed. The samples for the test are prepared in accordance with ASTM standard D1876-01, “Peel Resistance of Adhesives.” Two sheets of MYLAR® polyethylene terephthalate (PET) film stock (Hopewell, Va.) of thickness 10 microns are cut into strips 305 mm long and 25 mm wide. On one of the strips of PET a thin layer of the oligomer adhesive precursor of example 1 is spread. The adhesive precursor is uniformly spread on 241 mm of the film from one end. The other film strip is pressed onto the coated strip, spanning the length of the coating. The amount of the adhesive precursor spread on the bonded area is 0.3 g. Ten samples are made. The samples are then cured in a convection oven at 130° C. hours for four hours. After curing, the samples are allowed to cool to room temperature and conditioned at room temperature for 12 hours. The bonded strips of film are pulled apart by an Instron tester set at a constant head speed of 254 mm/min. In all ten cases the PET is deformed before the adhesive fails, indicating cohesive failure and showing that the strength of the adhesive is greater than the substrate. This demonstrates a sufficient adhesive material for package construction.

### Example 4

### Preparation of Glycerol-Citrate Oligomer Adhesive Precursor

To a beaker, 92.09 g glycerol (P&G Chemicals, Cincinnati, Ohio), one mole, and 0.48 g p-toluenesulfonic acid is added. The beaker is placed on plate situated under an overhead stirrer fitted with a 4-blade paddle mixing implement and a Brookfield viscometer. The glycerol p-toluensulfonic acid mixture is stirred and is heated to 60° C. One mole of citric acid (Aldrich, Milwaukee, Wis.), 192 g, is slowly added to the glycerol/p-toluensulfonic acid mixture while stirring. The mixture temperature is slowly raised 80° C. until a clear slightly straw-colored solution is formed. The temperature is then raised to 140° C. Some bubbling is noticeable at this time. The solution is stirred at 140° C. until a viscosity of 2 poise is indicated by the viscometer. The material is clear and straw-colored and easy to pour.

### Example 5

### Use of Glycerol Citrate Oligomer as an Adhesive for Paper

To test adhesive properties a simple adhesive test is completed. The samples for the test are prepared in accordance with ASTM standard D1876-01, “Peel Resistance of Adhesives.” The samples consist of two sheets of paper stock of weight 180 g/m² cut into strips 305 mm long and 25 mm wide. On one of the strips of paper a thin layer of the oligomer adhesive precursor of Example 4 is spread. The adhesive precursor is uniformly spread on 241 mm of the paper measured from one end of the strip. The other strip is pressed onto the coated strip, spanning the length of the coating. The amount of the adhesive spread on the bonded area is 0.3 g. Ten samples are made. The samples are then cured in a convection oven at 130° C. for four hours. After curing the strips are allowed to cool to room temperature and conditioned at room temperature for 12 hours. The bonded strips of paper are pulled apart by an Instron tester (Norwood, Mass.) set at a constant head speed of 254 mm/min. In all ten samples the paper is torn before the adhesive fails, indicating cohesive failure and showing that the strength of the adhesive is greater than the substrate. This makes for a sufficient adhesive material for package construction.

### Example 6

### Use of Glycerol Citrate Oligomer as an Adhesive for Polymers

To test adhesive properties a simple adhesive test is completed. The samples for the test are prepared in accordance with ASTM standard D1876-01, “Peel Resistance of Adhesives.” The samples consist of two sheets of MYLAR® polyethylene terephthalate (PET) film stock (Hopewell, Va.) of thickness 10 microns cut into strips 305 mm long and 25 mm wide. On one of the strips of PET a thin layer of the oligomer adhesive precursor of example 4 is spread. The material is spread on 241 mm of the film measured from one end and the film strip. The other film strip is pressed onto the coated strip, spanning the length of the coating. The amount of the adhesive spread on the bonded area is 0.3 g. Ten samples are made. The samples are then cured in a convection oven at 130° C. for four hours. After curing the samples are allowed to cool to room temperature and conditioned at room temperature for 12 hours. The bonded strips of PET are pulled apart by an Instron tester set at a constant head speed of 254 mm/min. In all ten cases the PET is deformed before the adhesive fails, indicating cohesive failure and showing that the strength of the adhesive is greater than the substrate. This makes for a sufficient adhesive material for package construction.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for bonding a material to a substrate comprising the steps of:
(a) providing a substrate to be bonded;
(b) providing an adhesive precursor comprising a reactive mixture, the reactive mixture selected from the group consisting of:
1) at least one polyhydric alcohol and reactant selected from the group consisting of: at least one organic polyacid; at least one organic anhydride; and combinations thereof;
2) a pre-polymer formed from a reactive mixture according to (1);
3) combinations of the reactive mixture in (1) and the pre-polymer in (2); and
4) combinations of the pre-polymer in (2) and reactants selected from the group consisting of: polyhydric alcohol; organic polyacid; organic anhydride; and combinations thereof;
(c) applying the adhesive precursor to at least one side of the substrate;
(d) placing the adhesive precursor in contact with a material to be bonded to the substrate; and
(e) reacting the adhesive precursor to form an adhesive as a product of ester condensation wherein the adhesive bonds the substrate to the material.

2. The method according to claim 1, wherein the polyhydric alcohol is selected from the group consisting of glycerol, glycol and combinations thereof.

3. The method according to claim 1, wherein the organic polyacid is selected from the group consisting of adipic acid, citric acid, maleic acid, succinic acid, polyacrylic acid and combinations thereof.

4. The method according to claim 1, wherein the anhydride is selected from the group consisting of succinic anhydride, maleic anhydride, phthalic anhydride and combinations thereof.

5. The method according to claim 1, wherein the reactive mixture further comprises monobasic acid, monoglyceride, diglyceride, or triglyceride.

6. The method according to claim 1, wherein the reactive mixture further comprises compounds having functional groups selected from the group consisting of acid groups, alcohol groups and combinations thereof, and further wherein the compounds are selected from the group consisting of oligomeric silicone, polyethylene glycol and combinations thereof.

7. The method according to claim 1, wherein the material comprises second substrate wherein the substrate is selected from the group consisting of films, sheets, nonwovens, paper and foams.

8. The method according to claim 1, wherein the material is selected from the group consisting of particles, granules, powders, and pellets.

9. A composite comprising a substrate, a material and an adhesive precursor disposed between the substrate and the material, the adhesive precursor comprising a reactive mixture selected from the group consisting of:
a) at least one polyhydric alcohol and reactant selected from the group consisting of: at least one organic polyacid; at least one organic anhydride; and combinations thereof;
b) a pre-polymer formed from a reactive mixture according to (a);
c) combinations of the reactive mixture in (a) and the pre-polymer in (b); and
d) combinations of the pre-polymer in (b) and reactants selected from the group consisting of: polyhydric alcohol; organic polyacid; organic anhydride; and combinations thereof;
wherein the adhesive precursor is reacted to form an adhesive as a product of ester condensation bonding the substrate and material.

10. The composite according to claim 9, wherein the polyhydric alcohol is selected from the group consisting of glycerol, glycol and combinations thereof.

11. The composite according to claim 9, wherein the organic polyacid is selected from the group consisting of adipic acid, citric acid, maleic acid, succinic acid, polyacrylic acid and combinations thereof.

12. The composite according to claim 9, wherein the anhydride is selected from the group consisting of succinic anhydride, maleic anhydride, phthalic anhydride and combinations thereof.

13. The composite according to claim 9, wherein the reactive mixture further comprises monobasic acid, monoglyceride, diglyceride, or triglyceride.

14. The composite according to claim 9, wherein the reactive mixture further comprises compounds having functional groups selected from the group consisting of acid groups, alcohol groups and combinations thereof, and further wherein the compounds are selected from the group consisting of oligomeric silicone, polyethylene glycol and combinations thereof.

15. The composite according to claim 9 wherein the substrate layer is selected from the group consisting of films, nonwovens, paper and foams and the material is selected from the group consisting of particles, granules, powders, and pellets.

16. The composite according to claim 9 wherein the substrate layer comprises a first substrate layer selected from the group consisting of films, nonwovens, paper and foams and the material comprises a second substrate selected from the group consisting of films, nonwovens, paper and foams.

17. A disposable article comprising components and an adhesive precursor disposed between at least two components, the adhesive precursor comprising a reactive mixture selected from the group consisting of:
a) at least one polyhydric alcohol and a reactant selected from the group consisting of: at least one organic polyacid; at least one organic anhydride; and combinations thereof;
b) a pre-polymer formed from a reactive mixture according to (a);
c) combinations of the reactive mixture in (a) and the pre-polymer in (b); and
d) combinations of the pre-polymer in (b) and reactants selected from the group consisting of: polyhydric alcohol; organic polyacid; organic anhydride; and combinations thereof;
wherein the adhesive precursor is reacted to form an adhesive as a product of ester condensation bonding the at least two components.

18. The disposable article according to claim 17, wherein the polyhydric alcohol is selected from the group consisting of glycerol, glycol and combinations thereof.

19. The disposable article according to claim 17, wherein the organic polyacid is selected from the group consisting of adipic acid, citric acid, maleic acid, succinic acid, polyacrylic acid and combinations thereof and wherein the anhydride is selected from the group consisting of succinic anhydride, maleic anhydride, phthalic anhydride and combinations thereof.

20. The disposable article according to claim 17 wherein the two components are selected from the group consisting of topsheet, backsheet, absorbent core, elastic waist features and leg cuffs.

21. Packaging comprising components and an adhesive precursor disposed between at least two components, the adhesive precursor comprising a reactive mixture selected from the group consisting of:
e) at least one polyhydric alcohol and reactant selected from the group consisting of: at least one organic polyacid; at least one organic anhydride; and combinations thereof;
f) a pre-polymer formed from a reactive mixture according to (a);
g) combinations of the reactive mixture in (a) and the pre-polymer in (b); and
h) combinations of the pre-polymer in (b) and reactants selected from the group consisting of: polyhydric alcohol; organic polyacid; organic anhydride; and combinations thereof;
wherein the adhesive precursor is reacted to form an adhesive as a product of ester condensation bonding the at least two components.

22. The packaging according to claim 21, wherein the polyhydric alcohol is selected from the group consisting of glycerol, glycol and combinations thereof.

23. The packaging according to claim 21, wherein the organic polyacid is selected from the group consisting of adipic acid, citric acid, maleic acid, succinic acid, polyacrylic acid and combinations thereof.
